(19) United States
(12) Patent Application Publication
(10) Pub. No.: US 2018/0140538 A1
(43) Pub. Date: May 24, 2018

(54) COMPOUND FOR ELIMINATING ODORS FOR HUNTING

(71) Applicant: MARLENE KERR, BROWNSTOWN TOWNSHIP, MI (US)

(72) Inventor: MARLENE KERR, BROWNSTOWN TOWNSHIP, MI (US)

(21) Appl. No.: 15/359,013

(22) Filed: Nov. 22, 2016

(51) Int. Cl. A61K 8/99; A61Q 15/00; A61Q 19/08; A61K 8/97; A61K 8/46; A61K 8/42; A61K 8/41; A61K 8/34; A61K 8/37; A61K 8/44; A61K 8/24; A61K 8/20; A61K 8/365; A61K 8/67

(52) U.S. Cl. CPC A61K 8/99; A61K 2800/596; A61Q 19/08; A61K 8/97; A61K 8/463; A61K 8/42; A61K 8/416; A61K 8/345; A61K 8/37; A61K 8/44; A61K 8/442; A61K 8/24; A61K 8/20; A61K 8/365; A61K 8/673; A61K 8/671; A61K 8/676; A61K 8/67; A61K 2800/74; A61Q 15/00

(57) ABSTRACT

A regimen for hunting and outdoor elements such as extreme cold or harsh weather conditions includes using a number of Aloe Vera-containing personal items in connection with a hunter's apparel and body to remove the hunter's body odor for improved hunting. The present invention also relates to the use of Saccharomyces Ferment containing compositions for the manufacture of an odor absorbing composition, which is safe for use on skin, for controlling environmental malodors on skin and for removing body odor. The personal items include lotion, cleansing cream, cleansing lotion, cleansing foam, shampoo and cleansing water.

### CROSS REFERENCE TO RELATED APPLICATIONS

Not applicable

### FEDERALLY SPONSORED RESEARCH AND DEVELOPMENT

Not applicable

### MICROFICHE

Not applicable

### BACKGROUND OF THE INVENTION

### (1) Field of the Invention

The invention relates to cosmetic compositions and methods for eliminating body odors, and more particularly pertains to a topically applied substance for eliminating body odors for hunting which comprise Cocamide MEA and Aloe extract.

### (2) Background of the Invention

Many attempts have been made in the form of cosmetic compositions for removing or reducing from the human body for individuals involve in various outdoor activities such as hunting, photographing or just observing wildlife. Given the keen sense of smell, sight, and sound that animals possess, utmost care and precaution must be exercised if one is to approach wildlife to engage in any of the above activities.

There have been many attempts and related patents to this endeavor, in which the following patents and applications are herein incorporated by reference for their supportive teachings, as follows:

US Patent Application No. 20100112025 to Farris Clinton W and Martin Myra A discloses a composition that is applied to the body of an individual, such as a hunter, a photographer, or an observer of wildlife, to inhibit the detection of human scent by wildlife. The composition acts as a scent inhibitor and not as a cover scent by greatly reducing or eliminating the exudation or loss of human skin cells that are the means by which wildlife detect the presence of a human being.

U.S. Pat. No. 5,780,020 issued to Liezl Gonzales Peterson et al. describes a powder, odor absorbing composition, which is safe for use on human skin comprising from about 0.1% to about 25%, by weight of the composition, of un-complexed cyclodextrin; and a powder carrier. The odor absorbing compositions of the present invention may also contain an additional odor controlling agent, selected from the group consisting of zeolites, activated charcoal, sodium bicarbonate, antimicrobial agents, and antiperspirants.

WO Patent Application No. 1998017240 to Toan Trinh et al. proposes an odor absorbing composition, which is safe for use on human skin comprising from 0.1% to 5%, by weight of the composition, of solubilized, water-soluble, un-complexed cyclodextrin; from 0.1% to 36%, by weight of the composition, of an oil phase selected from the group consisting of emollients, moisturizers, skin protectants, and mixtures thereof; an emulsifier; and an aqueous carrier.

Nonetheless, despite the ingenuity of the above compositions, there remains a need for a product that eliminates the odor of human scent and also moisturizes the skin while in the outdoor activities such as hunting, photographing or observing wildlife.

### SUMMARY OF THE INVENTION

In the view of the foregoing disadvantages inherent in the known types of cosmetic composition for eliminating body odors now present in the prior art, the present invention provides an improved cosmetic composition. As such, the general purpose of the present invention, which will be described subsequently in greater detail, is to provide a new and improved cosmetic composition which has all the advantages of the prior art and none of the disadvantages.

It is an objective of the present invention to provide an odor removing composition from skin and hair that comprises a genus Aloe plant extract and Saccharomyces Ferment, which helps in breakdown of odor-causing compounds caused from the bacteria in the body.

It is another objective of the present invention to provide an odor removing composition for skin and hair comprising water, a genus Aloe plant extract, Saccharomyces Ferment, Sodium Laurul Sulfate, Sodium Laureth, Cocamide MEA, Hydroxypropyltrimonium Honey, Sorbitol, Glycerin, Glycol Stearate, Tetrasodium, Cocamidopropyl Betaine, Phosphoric acid, Sodium Chloride, Citric Acid, D-Lactate and Vitamin-B12/A/C/K.

It is yet another objective of the present invention to provide an odor removing formulation for skin and hair in human that comprises topically applying a cosmetic or pharmaceutical composition to a skin surface.

It is still yet another objective of the present invention to provide an odor removing formulation for skin and hair that can be used by a hunter, a photographer or an observer of wildlife, and the general public to eliminate noisome odors.

It is still yet a further objective of the present invention to provide an odor removing formulation that moisturizes and hydrates skin and hair.

Still another objective of the present invention is to provide an odor removing formulation for application to the body of the hunter that imitates the light fragrances produced by nature.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### DETAILED DESCRIPTION OF THE ILLUSTRATED INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense, and the scope of the present invention is defined by the appended claims and their equivalents.

The present invention, which was made based on the fact that some natural ingredients effectively inhibit the formation of body odor of humans, in their formation stages, provides a body-odor inhibitory agent comprising Water, Aloe Extract, Saccharomyces Ferment, Sodium Laurul Sulfate, Sodium Laureth, Cocamide MEA, Hydroxypropyltrimonium Honey, Sorbitol, Glycerin, Glycol Stearate, Tetrasodium, Cocamidopropyl Betaine, Phosphoric acid, Sodium Chloride, Citric Acid, D-Lactate and Vitamin-B12/A/C/K.

A detailed description of essential and optional components of the present invention is given below.

Aloe Vera:—Aloe Vera is a succulent plant species of the genus Aloe. It mainly grows in tropical climates around the world and is cultivated for agricultural and medicinal uses. Because aloe Vera possesses excellent moisturizing capabilities it is commonly used as a daily skin cream. It is excellent for facial use, especially for women who use makeup frequently, as it effectively cleans and softens the skin. The moisturizing effect works for both men and women. The antimicrobial properties of aloe Vera also make it an excellent option for acne treatment and as an odor reducing agent.

Saccharomyces Ferment:—Saccharomyces is a member of the fungus family and various strains have been used for centuries in the pharmaceutical, cosmetic and food industry. Saccharomyces Ferment is a vegetable derived enzyme that helps break down odor-causing compounds caused from the bacterial breakdown of sweat. In essence, this enzyme provides bioconversion of a wide range of malodorous substances caused by perspiration.

Sodium Laurul Sulfate:—SLS is a detergent and surfactant which essentially means that it breaks surface tension and separates molecules in order to allow better interaction between the product and Skin/hair. This in turn creates a lather which makes products such as shampoo and body washes more effective cleansers.

Sodium Laureth:—Used primarily as a detergent cleansing agent. It could be derived from coconut. It's considered gentle and effective as typically used in cosmetics products (typically facial or body cleansers and shampoos). Sodium laureth sulfate is a surface-active agent or surfactant. All surfactants are partly water-soluble and partly oil-soluble. It is this quality that allows oil and water, which normally don't mix together, to become dispersed. Sodium laureth sulfate gives thick rich foam and it cleanses the Skin/hair. Its thorough action dissolves dirt and grease effectively.

Cocamide MEA:—Cocamide MEA is derived from the fatty acids from coconut oil and monoethanolamine (MEA). In cosmetics and personal care products, Cocamide MEA is used in the formulation of bath soaps and shampoo. Cocamide MEA is a mixture of ethanolamines of fatty acids derived from coconut oil. This cosmetic ingredient functions as a surfactant—foam booster and an aqueous viscosity-increasing agent. It allows the hands to work shampoo through the hair. This helps the mechanical removal of grease and odor. Cocamide MEA suits opaque, thick formulae, as it increases product viscosity. Cocamide MEA increases foaming capacity and/or stabilizes foam. Cocamide MEA is also used to thicken the aqueous (water) portion of cosmetic and personal care products.

Hydroxypropyltrimonium Honey:—Hydroxypropyltrimonium Honey or Honeyquat is substantive and moisturizing and with its low molecular weight, able to penetrate hair and skin to provide its moisturizing benefits over time while providing shine and luster. Honeyquat will improve wet-combability of the hair and reduce static buildup when dry combing. It is made by modifying honey with a low molecular weight quaternary derivative to make it practically non-sticky. This humecant binds environmental moisture to itself, increasing the miniaturization of skin and hair. It improves the wet-combability of hair and helps to reduce static build up on dry hair.

Sorbitol:—Sorbitol is a sugar alcohol naturally found in fruits. Its chemical properties allow it to have multiple actions within the body. Sorbitol is a sugar alcohol derived from fruits such as pears, peaches, prunes, cherries, plums, berries and apples and seaweed although most products currently use corn-derived Sorbitol. It is used primarily as a humectant in cosmetics and personal care products, although it is also a fragrance ingredient and flavoring agent (thanks to its sweet flavor). As a humectant, Sorbitol prevents moisture loss by pulling water by osmosis from the air, hydrating skin; however, when used in extremely dry conditions, Sorbitol can instead whisk moisture out of the skin and leave it dry or damaged.

Glycerin:—Also called glycerol or glycerine, glycerin is present in all natural lipids (fats), whether animal or vegetable. It can be derived from natural substances by hydrolysis of fats and by fermentation of sugars; it also can be synthetically manufactured. Glycerin is an organic compound known more formally as glycerol. Its common sources are animal fat and vegetable oil. Glycerin is a clear, odorless liquid at room temperature, and it has a sweet taste. It's most commonly used in soap and is also a common ingredient in many pharmaceuticals. Natural glycerin is not a chemical or alcohol; it is derived from plant-based oils. It has hygroscopic properties, meaning it absorbs moisture and, if left alone, would absorb up to 20% of its weight in water.

Glycol Stearate:—Glycol stearate is a combination of carbon, hydrogen and oxygen atoms. The substance is a product of stearic acid, a common fatty acid that is found in both plants and animals. The Glycol Stearate ingredients consist of mixtures of the mono- and di esters of ethylene glycol and triple-pressed stearic acid. Stearic acid is a common fatty acid. Glycol stearate has the ability to smooth and soften the skin tissue when applied topically. Its fatty, thick texture allows it to fill in the tiny spaces that exist between the protein fibers. This makes glycol stearate what is known as an emollient. Especially beneficial for dry skin, glycol stearate is found in a wide variety of moisturizers, body lotions, body butters and even hair conditioners.

Tetrasodium:—Tetrasodium ETDA (which stands for ethylenediaminetetraacetic acid) is a water-soluble ingredient used as a “chelator,” which means it binds to certain mineral ions to inactivate them. Through this action, it can prevent the deterioration of cosmetic and personal care products, as it stops the growth of mold and other microorganisms. Tetrasodium EDTA also helps maintain clarity, protect fragrance compounds, and prevent rancidity. One of its main uses it to help personal care products work better in hard water.

Cocamidopropyl Betaine:—CocoBetaine (Cocamidopropyl Betaine) is a, 30% active, aqueous surfactant solution, from Coconut Oil. CocoBetaine (Cocamidopropyl Betaine) is used in shampoos, bubble baths and liquid hand soaps, as a secondary surfactant in cleansing systems where it will help to build viscosity and offers a smooth feel. CocoBetaine (Cocamidopropyl Betaine) provides good foaming, and foam liquid stabilization, with excellent wetting properties. As a surfactant, it's mostly used in bath products because it gently cleanses the skin/hair by helping water to mix with oil and dirt so that they can be rinsed off. On top of that, its pH range contributes a mild germicidal effect, which also makes it an effective, yet mild, cleansing agent for personal sanitary products.

Phosphoric acid:—Phosphoric Acid is a colorless, odorless phosphorus-containing inorganic acid. Phosphoric acid is a clear, colorless, odorless liquid with a syrupy consistency. Phosphoric acid is used as a pH adjuster in cosmetics and skin-care products.

Sodium Chloride:—Sodium Chloride is a metal halide composed of sodium and chloride with sodium and chloride replacement capabilities. Used primarily as a binding agent in skincare products and occasionally as an abrasive in scrub products. Perhaps the most common use of sodium chloride in cosmetics is as a thickening agent. Sodium Chloride is commonly used to thicken the water (also known as aqueous) phase of shampoos, body washes, and non-soap facial cleansers.

Citric Acid:—Citric acid is a natural, weak organic acid that is found in many fruits and vegetables, especially citrus. Citric acid acts as a natural skin scrubber, and is very effective for de-pigmenting skin. Citric acid is very beneficial in curing the sun tanned skin. Citric acid is an antioxidant that reduces the process of aging.

D-Lactate:—Lactate is the anion produced from lactic acid. When lactic acid is dissolved in water, it tends to dissociate, and produces a lactate ion and a proton. lactate increases the moisture content of skin and hair even when it is then rinsed off. This makes sodium lactate a useful ingredient in skin washes and toners, which are designed to be used as intermediate steps in a beauty routine. Sodium lactate is also effective as a hydrating ingredient in shampoos.

Vitamin B12/A/C/K:—Vitamin is chemically a group of organic compounds which are essential for normal growth and nutrition and are required in small quantities in the diet because they cannot be synthesized in the body through the various synthesis pathways in the body. Vitamin B 12, also known as Cobalamin, is one of the 8 vitamins found in vitamin B complex. Vitamin B-12 provides great benefit to the skin, helping to repair skin cells and keep your skin looking its best. Getting the right mix of B vitamins can help conditions ranging from dryness and redness to acne and other blemishes. A Vitamin A, or retinol, is one of the most widely acknowledged nutrients for healthy skin. Vitamin C is also an effective antioxidant. This means that it can also help you battle the signs of skin aging. Vitamin C also stimulates the production of collagen in skin. Collagen is the protein responsible for making skin supple. Vitamin K is essential in aiding the body's process of blood clotting, which helps the body heal the areas bruised during surgery. Topical creams with vitamin K can also help treat unsightly skin irritations, such as spider veins, stretch marks, scars, and dark circles under the eyes.

The odor removing cosmetic composition skin or hair of the invention includes the aforementioned components and may optionally include well-known components depending on a particular application, usage or form of the cosmetic composition. For example, there may be added water, Aloe plant extract; at least one extract from a probiotic microorganism; at least one anionic compound from a surfactant; at least one fatty acid from coconut oil; a conditioning agent; at least one sugar alcohol from Polyol; at least one self-emulsifying agent; a chelating agent; at least one organic compound from coconut oil; at least one inorganic compound from mineral acid; a binding agent; at least one at least one anti-inflammatory ingredient from organic tribasic acid; at least one ester from lactic acid; and a vital nutrient etc. as long as they do not compromise the objects of the invention.

Example of the invention is given below by way of illustration and not by way of limitation.

### EXAMPLE

TABLE 1
Odor removing formulation for Skin and Hair in Humans containing
Aloe Vera extract and Saccharomyces Ferment treatment is
prepared based on the constitutions as follows.

| S. No. | Composition | wt % |
|---|---|---|
| 1. | Water | 70 |
| 2. | Aloe Extract | 5 |
| 3. | Saccharomyces Ferment | 1 |
| 4. | Sodium Laurul Sulfate | 5 |
| 5. | Sodium Laureth | >1 |
| 6. | Cocamide MEA | 5 |
| 7. | Hydroxypropyltrimonium Honey | 1 |
| 8. | Sorbitol | 2 |
| 9. | Glycerin | 3 |
| 10. | Glycol Stearate | 1 |
| 11. | Tetrasodium | 1 |
| 12. | Cocamidopropyl Betaine | >1 |
| 13. | Phosphoric acid | 1 |
| 14. | Sodium Chloride | 2 |
| 15. | Citric Acid | >1 |
| 16. | D-Lactate | >1 |
| 17. | Vitamin-B12/A/C/K | >1 |

In the most preferred embodiment of the invention, the amount of the active ingredients Aloe Vera extract and Saccharomyces Ferment is in the range of from 0.5-5% and 0.2-1% respectively by weight of the composition.

Procedure:—All Aloe Vera extract and Saccharomyces Ferment are dissolved in part of water (70% water) from the batch. Cocamide MEA is then added to it with mixing to form the pre-blend. All remaining ingredients are then mixed in a separate tank. The pre-blend is then added to the main batch with mixing. A syrupy cleanser product is obtained that contains >1% of NaCl.

Use of the Composition:—The composition according to the invention is intended primarily as a product for topical application for removing odor from human skin and hair, especially as an agent for controlling or preventing odor forming components and for improving skin's radiance and clarity. In use, a quantity of the composition, for example from 1 to 100 ml, is applied to exposed areas of the skin, from a suitable container or applicator and, if necessary, it is then spread over and/or rubbed into the skin or hair using the hand or fingers or a suitable device.

Product Form and Packaging:—The cosmetic composition for removing odor from skin and hair for human is not particularly limited with regard to the formulation thereof and the formulation may be determined appropriately depending on purposes. The cosmetic composition may be provided as one or more formulation selected from a group consisting of lotion, essence, cream, ointment, gel, pack, patch, spray, powder foundation, emulsion foundation, conceal stick, hand or foot lotion, hand or foot cream, hand or foot oil, hand or foot essence, hand or foot cleanser, soap, cleansing cream, cleansing lotion, cleansing foam, shampoo and cleansing water, but is not limited thereto.

Advantageous Effects:—The Odor removing composition from Skin and Hair in Humans according to the present disclosure provides the effect of eliminating odor from skin and hair, moisturizing skin, anti-inflammation, anti-microbial, preventing skin aging, anti-oxidation, improving acne, improving atopic dermatitis, relieving skin irritation, maintaining skin homeostasis and relieving or improving skin allergy, and is useful in wide applications including, for example, cosmetics or pharmaceuticals.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1. A cosmetic composition for removing body odor from human, comprising:
a) a genus Aloe plant extract;
b) at least one extract from a probiotic microorganism;
c) at least one anionic compound from a surfactant;
d) at least one fatty acid from coconut oil;
e) a conditioning agent;
f) at least one sugar alcohol from Polyol;
g) at least one self-emulsifying agent;
h) a chelating agent;
i) at least one organic compound from coconut oil;
j) at least one inorganic compound from mineral acid;
k) a binding agent;
l) at least one at least one anti-inflammatory ingredient from organic tribasic acid;
m) at least one ester from lactic acid; and
n) a vital nutrient.

2. The cosmetic composition as in claim 1, wherein said genus Aloe plant extract is from the plant Aloe Vera.

3. The cosmetic composition as in claim 1, wherein said microorganism is yeast.

4. The cosmetic or pharmaceutical composition as in claim 3, wherein said yeast comprises Saccharomyces cerevisiae.

5. The cosmetic composition as in claim 4, wherein said anionic compound is selected from the group consisting of Sodium Laurul Sulfate and Sodium Laureth.

6. The cosmetic or pharmaceutical composition as in claim 5, wherein said fatty acid from coconut oil is Cocamide monoethanolamine (MEA).

7. The cosmetic composition as in claim 6, wherein said conditioning agent is Hydroxypropyltrimonium Honey.

8. The cosmetic composition as in claim 7, wherein said sugar alcohol from Polyol is selected from the group consisting of Sorbitol and Glycerin.

9. The cosmetic composition as in claim 8, wherein said self-emulsifying agent said is Glycol Stearate.

10. The cosmetic composition as in claim 9, wherein said chelating agent is Tetrasodium.

11. The cosmetic or pharmaceutical composition as in claim 10, wherein said organic compound from coconut oil is Cocamidopropyl Betaine.

12. The cosmetic composition as in claim 11, wherein said inorganic compound from mineral acid is Phosphoric acid.

13. The cosmetic composition as in claim 12, wherein said binding agent is Sodium chloride.

14. The cosmetic composition as in claim 13, wherein said anti-inflammatory ingredient is Citric acid.

15. The cosmetic composition as in claim 14, wherein said ester from lactic acid is D-Lactate.

16. The cosmetic composition as in claim 15, wherein said vital nutrient is selected from the group consisting of Vitamin-B12, Vitamin-A, Vitamin-C and Vitamin-K.

17. The cosmetic composition as in claim 16, wherein said cosmetic composition is formulated in the form selected from the group consisting of lotion, essence, cream, ointment, gel, pack, patch, spray, powder foundation, emulsion foundation, conceal stick, hand or foot lotion, hand or foot cream, hand or foot oil, hand or foot essence, hand or foot cleanser, soap, cleansing cream, cleansing lotion, cleansing foam, shampoo and cleansing water.
